(19) United States
(12) Patent Application Publication
(10) Pub. No.: US 2009/0099499 A1
(43) Pub. Date: Apr. 16, 2009

(54) INTELLIGENT SEQUENTIAL ILLUMINATING DEVICE FOR PHOTODYNAMIC THERAPY

(76) Inventors: Antun Persin, Zagreb (HR); Hrvoje Zorc, Zagreb (HR); Anton Radman, Zagreb (HR); Vitomir Stanisic, Zagreb (HR); Nenad Agatic, Zagreb (HR); Dunja Soldo Roudnicky, Zagreb (HR); Kreso Tisaj, Zagreb (HR); Martin Loncaric, Zagreb (HR)

Correspondence Address: ST. ONGE STEWARD JOHNSTON & REENS, LLC, 986 BEDFORD STREET, STAMFORD, CT, 06905-5619, US

(21) Appl. No.: 12/252,785

(22) Filed: Oct. 16, 2008

Related U.S. Application Data
(63) Continuation of application No. PCT/HR2007/000012, filed on Apr. 19, 2007.

(30) Foreign Application Priority Data
Apr. 19, 2006 (HR) P20060149A

(51) Int. Cl. A61N 5/06

(52) U.S. Cl. 604/20

(57) ABSTRACT

Intelligent sequential illuminating device for photodynamic therapy of surface tumors that comprises: the module for illumination and detection, module for signal processing and control and module for power supply, of which the module for illumination and detection consists of the violet light emitting diodes (405) and the red light emitting diodes (640), where the violet light emitting diodes serve for fluorescence excitation of the photo reactive agent and the red light emitting diodes have twofold purpose: for emission and therapeutic red light and for detection of the red fluorescent light caused by illumination of the violet light emitting diodes. The module for signal processing and control manages a work of the light emitting diodes so that the violet light emitting diodes are activated into the determined sequences during which the red light emitting diodes measure the level of ppix fluorescence. Depending on the measured fluorescent intensity, the red light emitting diodes are activated between the pulses of the violet light emitting diodes.

### CROSS-REFERENCE TO RELATED APPLICATIONS

The present application is a continuation of pending International patent application PCT/HR2007/000012, filed Apr. 19, 2007, which designates the United States and claims priority from Croatian patent application no. P20060149A, filed Apr. 19, 2006, the content of which is incorporated herein by reference.

### TECHNICAL FIELD

This invention concerns the intelligent sequential illuminator for photodynamic therapy of malignant and non-malignant skin diseases using protoporphyrin IX (abbr. ppix) generated by means of 5-aminolevulinic acid (abbr. 5-ALA). During therapy treatment, the illuminator automatically measures a condition of ppix in tissue, and in relation to that, the illuminator determines a therapeutic regime of photodynamic therapy.

International Classification:

A61 B6/00 Device and Apparatus Applicable to Both Therapy and Diagnosis A61 B6/06 Using Light (A61 N5/01 Takes Precedence)

G01 N21/64 System in Which the Material Investigated is Exited Whereby it Emits the Light or Causes a Change in Wavelength of the Incident Light G01 N21/63 Optically Excited G01 N21/64 Fluorescence, Phosphorescence

### TECHNICAL PROBLEM

Photodynamic therapy (abbr. PDT) is a process at which partake: a photo reactive agent that accumulates in the tissue diseased, photosensitizing light and oxygen that comes into an interactive area. The photo reactive agent is being excited at this interaction by light and transfers its excitation onto molecular oxygen. The molecular oxygen is transformed into reactive singlet oxygen. As the photo reactive agent is accumulated in a diseased cell, the singlet oxygen damages the cell. In this way, the diseased tissue is destroyed selectively. In addition to direct damaging through the singlet oxygen and radicals that cause necroses of tissue, there is also a mechanism of self destruction at photodynamic therapy—the apoptosis of the diseased cells. A ratio between necrosis and apoptosis depends on the type of the photo reactive agent, type of the diseased cells and intensity and illumination dose.

The process efficiency depends on an intensity and wavelength of the light delivered. A dynamics of the photodynamic therapy changes as Well during therapeutic treatment. The generated singlet oxygen also destroys the molecules of the photo reactive agent herewith reducing its concentration.

Oxygen is also depleted during the process, its concentration reduces, and with this an efficiency of a therapeutic process is also reduced.

In general, the efficiency of photodynamic process depends on oxygen supply and a formation rate of the photo reactive agent.

The efficacy of the process is achieved with an optimum choice of the light intensity and a wavelength of the light delivered. In the case when the photo reactive agent protoporphyrin IX (ppix) is applied, 5-aminolevulinic acid (5-ALA) is used as a starting material. During metabolic process, 5-ALA undergoes its transformation into the ppix. A control of ppix concentration during photodynamic therapy is being monitored through its fluorescence. A therapeutic excitement of ppix is performed with the light at the wavelengths of 620 nm to 660 nm and the fluorescent excitement within the waveband of 395 nm to 410 nm.

A result of the dynamics of the photodynamic process is a reduction of ppix concentration during therapeutic illumination, i.e. the concentration of ppix is increased—generated again after stopping the therapeutic illumination. A given optimal concentration of ppix is achieved by choosing an intensity and/or duration of illumination. By measuring the ppix fluorescence during therapeutic illumination, one can determine (defined) a level of ppix. Stopping the illumination and waiting until the ppix concentration is generated again, it is possible to maintain the concentration at the given level during phototherapeutic process. Too high intensity of the therapeutic light will bleach out protoporhyrin IX, or oxygen supply will be insufficient to generate singlet oxygen. Too low intensity will not give sufficient efficacy. A measurement of the level of PpIX concentration along with the control of the illumination gives maximum therapeutic efficacy. This patent describes an apparatus and a method by which the illumination can be kept so that a photodynamic protocol is applied in an optimal regime. Hereby, the protocol enables the two regimes of illumination: a fractional illumination and a metronomic working regime.

The patent solves a technical problem of the optimum illumination taking into account a condition of the tissue and a real concentration of ppix.

The second problem that occurs at photodynamic therapy is spatial selectivity of illumination. Considering that ppix accumulates in the healthy tissue too, there is a risk that by illuminating the areas with the healthy tissue, these areas will also be damaged during photodynamic process. This problem has been solved by means of photodynamic shields, which have prevented the illumination of the healthy tissue. The problem is that an area of the malignant lesion is difficult to define technically. The patent solves the problem so that the fluorescence of ppix is measured at the given points. In this way, it is possible to determine the areas that fluoresce. This is the area where ppix is accumulated, and this is the area where the concentration of diseased cells does exist.

### STATE OF THE ART

Illuminators for photodynamic therapy and devices for fluorescent diagnostics have been described in patent bases and publications:

2006.
1. US 2006 0004347 (2006-01-05) Altshuler Gregory B.: Methods and Products for Producing Lattices of EMR—Treated Islets in Tissue and Uses therefore
2. U.S. Pat. No. 6,986,782 (2006-01-17) Chen James: Ambulatory Photodynamic Therapy.
3. WO 2006 0050 88 (2006-01-19) Torsier Walter: Device for Photodynamicaly Treating Diseases of the Tissue and or Organs
4. U.S. Pat. No. 6,991,644 (2006-01-31) Spooner Greg: Method and System for Controlled Spatially-Selective Epidermal Pigmentation Phototherapy with UVA LEDs
5. WO 2006 012737 (2006-02-09) Jungwirth Paul “Lighting System Including Photonic Emission and Detection Using Light-emitting Elements”
6. U.S. Pat. No. 7,001,413 (2006-02-21) Butler Glenn: “Methods and Apparatus for Light Therapy”

2005.
1. U.S. Pat. No. 6,860,896 (2005-03-01) Leland S. Leber: Therapeutic Methods and Apparatus
2. US 2005 0049582 (2005-03-03) Leonard C. De Benedictis: Method and Apparatus for Fractional Phototherapy of Skin
3. US 2005 0075 703 (2005-04-07) Larsen Eric: Photodinamic Stimulation Device and Methods
4. US 2005 0080475 (2005-04-14) Zelickson Brian: Device and Methods for Treatment of External Surface of the Body Utilizing a Light Emitting Container
5. US 2005 0087750 (2005-04-28) Braddell Jules: LED Array
6. US 20050085 455 (2005-04-28) Chen James: Photodynamic Therapy for Local Adipocyte Reduction
7. WO 2005 039699 (2005-05-06) Williams Christopher: Apparatus for Illuminating a Zone of Mammalian Skin
8. U.S. Pat. No. 6,899,723 (2005-05-31) James Chen: Transcutaneus Photodynamic Treatment of Targeted Cells
9. US 20050151489 (2005-07-14) Lys, Ihor A. Mueller Gorge: Market place illumination methods and apparatus
10. 2005 01 77093 (2005-08-11) Barry Hart M.: Joint Tissue Inflammation Therapy and Monitoring Device
11. US 2005 01 82460 (2005-08-18) Kent Marska, Lynch Ron: Light Therapy Device
12. U.S. Pat. No. 6,936,885 (2005-08-30) Shane Harrak: Bendable High Flux LED Array
13. U.S. Pat. No. 6,955,684 (2005-10-18) Savage Jr. Henry: Portable Light Delivery Apparatus and Method
14. CA 2.465.051 (2005-10-23) Dickey Dwayne, Moore Ronald: Switched Photodynamic Apparatus
15. US 2005 0265029 (2005-12-01) Keneth A. Epstein: LED Array System

2004.
1. WO 2004 017886 (2004-04-03) Williams Jeffrey: The Pad Like Device for Use during Phototherapy Treatment
2. WO 2004 043543 (2004-05-27), Altshuler Gregory: Apparatus for Performing Optical Dermatology
3. U.S. Pat. No. 6,743,249 (2004-06-01) Philip G. Alden: Treatment Device for Photodynamic Therapy and Method for Making Same
4. US 2004 0116913 (2004-06-17) Pilcher Kenneth A.: System for Treatment of Acne Skin Condition Using a Narrow Band Light Source
5. WO 2004 05 2238 (2004-06-24) Holloway Paul: Phototherapy Bandage
6. US 2004 012 7961 (2004-07-01) Whitehurst Colin: Therapeutic Light Source and Method
7. US 2004 138726 (2004-07-15) Savage Henry: Portable Light Delivery Apparatus and Methods for Delivering Light to the Human Body
8. US 2004 0166146 (2004-08-26) Holloway Paul H.: Phototherapy bandage
9. U.S. Pat. No. 6,800,086 (2004-10-05) H. Andrew Strong: Reduced Fluence Rate PDT
10. US 2004 0197267 (2004-10-07) Robert Black: In Vivo Fluorescence Sensor Systems and Related Methods Operating In Conjunction with Fluorescent Analytes
11. US 2004 0215292 (2004-10-28) Chen James: Photodynamic Treatment of Targeted Cells
12. U.S. Pat. No. 6,811,563 (2004-11-2) Savage Jr. Henry: Portable Light Delivery Apparatus and Methods for Delivery Light To the Human Body
13. WO 2004 096 343 (2004-11-11) Molina Sherry: Light and Magnetic Emitting Mask
14. WO 2004 100 789 (AU 2004238182), EP 1624803 (2004-11-25) Soto Thompson Marcelo, Anderson Engels Stephan: System and Method for Therapy and Diagnostic Comprising Optical Component for Distribution of Radiation
15. WO 2004 100 789 (2004-11-25) Soto Thompson Marcelo: System and Method for Therapy and Diagnosis Comprising Optical Components for Distribution Radiation
16. JP 2004358063 (2004-12-24) Tani Hiromachi: Therapeutic Attached Object

2003.
1. US 2003 009205 (2003-01-09) Biel Merrill A.: Treatment Device for Topical Photodynamic Therapy and Method Using Same
2. U.S. Pat. No. 6,528,954 (2003-03-04) Lys Ihor: Smart Light Bulb
3. US 2003 007628 (2003-04-24) Morgan Frederick, Lys Ihor: Diffuse Illuminator System and Methods
4. U.S. Pat. No. 6,521,118 (2003-05-27) Urs Utzinger: Combined Fluorescence and Reflectance Spectroscopy
5. US 2003 01 00838 (2003-05-29) Ly Ihor: Precision Illumination Method and Systems
6. US 2003 01144 34 (2003-06-19) Cheng James: Extended Duration Light Activated Cancer Therapy
7. CN 256 0841 Y (2003-07-16) Deng Jingquan: Light Base Patch Device
8. US 2003 0167033 (2003-09-04) System and Methods for Photodynamic Therapy
9. WO 0303076013 (2003-09-18) Azarenko Aleksei Nikolaevich: Device for Phototherapy
10. US 2003 0198450 (2003-10-23) Pafchek Robert M: Optoelectronic Device Having A Direct Mask Formed Thereon and Method of Manufacture Thereafter
11. WO 2003 03 098 707 (2003-11-27) Braddell: Led Array

2002.
1. U.S. Pat. No. 6,340,868 (2002-01-22) Lys Ihor, Mueller George: Illumination Components
2. US 2002 0087 205 (2002-07-04) Chen James: Transcutaneous Photodynamic Treatment of Targeted Cells
3. U.S. Pat. No. 6,459,919 (2002-10-01) Lys Ihor, Mueller George: Precision Illumination Methods and Systems

2001.
1. U.S. Pat. No. 6,231,593 (2001-05-15) Maseral Peter M: Patch, Controller and Method for Photodynamic Therapy of Dermal Lesion
2. WO 20010135997 (2001-05-25), Allison Beth Anne: Use of Low-Dose PDT to Inhibit Restenosis

2000.
1. U.S. Pat. No. 6,011,563 (2000-01-04) Fournier R: Computer Controlled Photoirradiation during Photodynamic Therapy
2. U.S. Pat. No. 6,048,359 (2000-04-11) Biel Merill: Spatial Orientation and Light Source and Method of Using Same for Medical Diagnosis and Photodynamic Therapy
3. U.S. Pat. No. 6,096,066 (2000-08-01) Chen James: Conformal Patch for Administering Light Therapy to Subcutaneous Tumors

1999.
1. WO 099 100 46 (1999-03-04) Biel Merril A: Treatment Device for Topical Photodynamic Therapy and Method of Making Same
2. U.S. Pat. No. 5,955,490 (1999-09-21) James C. Kennedy: Photochemotherapeutic Method Using 5-Aminolevulinic Acid and Other Precursors of Endogenous Porphyrins

1998.
1. U.S. Pat. No. 5,800,478 (1998-09-01) James Chen: Flexible Microcircuits for Internal Light Therapy
2. U.S. Pat. No. 5,766,234 (1998-06-16) James Chen: Implanting and Fixing a Flexible Probe for Administering a Medical Therapy at a Treatment Site within a Patient Body

1997.
1. WO 970 4836 (1997-02-13) Meserol: Patch, Controller and Method for the Photodynamic Therapy of a Dermal Lesion
2. U.S. Pat. No. 5,698,866 (1997-12-16) Doivon Daniel R: Uniform Illuminator for Phototherapy
3. U.S. Pat. No. 5,616,140 (1997-04-01) Marvin Preseot: Method and Apparatus for Therapeutic Laser Treatment

1996.
1. U.S. Pat. No. 5,489,279 (1996-02-06) Maserol Peter: Method of Applying Photodynamic Therapy to Dermal Lesion
2. U.S. Pat. No. 5,505,726 (1996-04-09) Maseral Peter: Article of Manufacture for the Photodynamic Therapy of Dermal Lesion
3. WO 1996 1821 (1996-06-13) Ignatius Ronald W: Arrays of Optoelectronics Device and Method of Making Same

1995.
1. U.S. Pat. No. 5,445,608 (1995-08-29) Chen James: Method and Apparatus for Providing Light Activation Therapy
2. U.S. Pat. No. 5,474,528 (1995-12-12) Maseral Peter: Combination Controller and Patch for the Photodynamic Therapy of Dermal Lesion

1994.
1. WO 1994 15666 (1994-06-21), Lytlea Charles: Light Emitting Diode Source for Photodynamic Therapy
2. U.S. Pat. No. 5,358,503 (1994-10-25) Bertwell Dale E.: Photo-thermal Therapeutic Device and Method

1993.
1. WO 1993 21842 (1993-11-119 Bower Robert: High Power Light-Emitting Diodes for Photodynamic Therapy

Photodynamic therapy by means of protoporphyrin IX (ppix) has been published in the following literature:
Z. Malik, H. Lugaci (1987) “Destruction Of Erythroleukemic Cell by Photoactivation of Endogenous Porphyrin”, Br. J. Cancer 1987, 56 (589-595)
Kennedy J C, Pottier R H, P/os D C “Photodynamic Therapy with Endogenous Protoporphyrin IX: Basic Principles and Present Clinical Experience”: J. Photochem Photobiol B. Biol 1990., 6_J43-148

Ppix fluorescence has been published in the following literature:
F. H. J. Figge G. S. Weiland C J. Manganiello: Cancer Detection and Therapy, Affinity of Neoplastic, Embryionic and Traumatized Tissue for Porphyrin and Metaloporphyrin, Proc. Soc. Exp. Biol. Med. 1948, 68 (8640-641)
M. Kriegmair, R. Baumgartner, R. Knueckel, H. Stepp, F. Hofstaedter: Detection of Early Blader Cancer by 5-Aminolevulinic Acid Induced Fluorescence, J. Urol. (1996) 155, 105-110
C. Fritsch, P. M. Becker-Wegerich, H. Menke, T. Ruzicka and others: Successful Surgery of Multiple Recurrent Basal Cell Carcinoma Guided by Photodynamic Diagnosis: Aest. Plast. Surg. 1997, 21, 437-439

Efficacy enhancement by fractional illumination has been published in the following literature:
K. P. Nielsen, Asta Juzeniene, Petras Juzenas, Knut Stamnes: Choice of Optimal Wavelength for PDT: The Significance of Oxygen Depletion; Photochemistry and Photobiology and Photobiology, 2005, 81 (1190-1194)
L. B. Oberdaner, K. Plaetzer, T. Kiesslich, B. Krammer: Photodynamic Treatment with Fractionated Light Decreases Production of Reactive Oxygen Species and Cytotoxicity in Vitro via Regeneration of Glutathione: Photochemistry and Photobiology 2005, 81, (609-613)
I. van den Boogert, H. J. van Staveven . . . , Fractionated Illumination for Oesophageal ALA-PDT: Effect on Blood Flow and Ppix formation, Laser in Medical Science (2001), 16-1-(16-25)
Dominic J. Robinson at all: Dose and Timing of the Firsts Light Fraction in Two-Fold Illumination Schemes for Topical ALA-mediated Photodynamic Therapy of Hairless Mouse Skin, Photochemistry and Photobiology, 2003, 77 (3), 319-329
Seiicki Linuma, Kevin T. Schomacher, Georges Wagnieres . . . : In Vivo Fluence Rate and Fractionated Effects on Tumor Response and Photobleaching: Photodynamic Therapy with Two Photosensitizers in an Orthotopic Rat Tumor Model; Cancer Research (1999), 59, (6164-6170)
M. A. Herman i dr.: Effect of Fractionated 5-Aminolevulinic Acid Administration on Tissue Levels of Protoporphyrin in Vivo; Jour. Photochem. Photobiol. B. Biology 1997, 40 (107-110)
Henderson B W Gollnick S O, Snyder J W, Busch T M, Kousis P C, Cheney R T, Morgan J: Choice of Oxygen-Conserving Treatment Regimen Determines the Inflammatory Response and Outcome of Photodynamic Therapy of Tumors, J. Cancer. Res. 2004, 15 64 (6) (2120-2126)
Nynke van der Veen, Henritte S. De Brujin and Willem M. Star: Photobleaching During and Re-Appearance after Photodynamic Therapy of Topical ALA-Induced Fluorescence in UVB-treated Mouse Skin, Int. J. Cancer (1997), 72, (110-118)
Simone Mueller, Heinrich Walt and al.: Enhanced Photodynamic Effect Using Fractionated Laser Light, Journal Photochem. Photobiol., Biol. (1998), 42 (67-70)
Henrieta S. de Bruijn and all: Improved of Systemic 5-aminolevulinic Acid Based Photodynamic Therapy in Vivo Using Light Fractionation With A 75-Minute Interval, Cancer Research (1999) 59,
Monique R. Thissen and all: Ppix Fluorescence Kinetics and Increased Skin Damage after Intracutaneous Injection of 5-Aminolevulinic Acid and Repeated Illumination, Journ. Invest. Dermatol, 2002, 118 (239-245)
D. J. Robinson and all: Dose and Timing of the First Light Fraction in Two-fold Illumination Schemes for Topical ALA-medicated Photodynamic Therapy of Hairless Mouse Skin (Photoch. Photobiol (2003) 77 (3) 319-329
Hiroaki Togashi, Mastaka Nehara, Hizasumi Ikeda, Tsugio Inokuci: Fractionated Photodynamic Therapy for a Human Oral Squamus Cell Carcinoma, Xenograft Oral Oncology, 2006 (In. Press)
Patricia Soo-Ping Thong, Frank Watt, Min Qin Ren, Puay Hoon Tan, Khee Chee Soo, Malini Olivo: Hypericin-photodynamic Therapy (PDT) Using Alternative Treatment Regime Suitable for Multifraction PDT, Journ. Photochem. Photobiol., B Biol (2006) 82 (1-8)

A principle of the metronomic photodynamic therapy has been published in:
Tao Xu, Yingxing Li, Xing Wu: Application of Lower Fluence Rate For Less Microvasculature Damage And Greater Cell-Killing During Photodynamic Therapy; Laser in Medical Science (2005) 19, 257-261
R. B. Veenhuizern and F. A. Stewart: The Importance of Fluency Rate in Photodynamic Therapy; Is There a Parallel with Ionizing Radiation Dose-Rate Effects? Radiotherapy and Oncology (1995) 37-2-131-135
Steven L. Jacques, Sergio Furuzava, Tom Rodrigez: PDT with ALA/PPIX is Enhanced by Prolonged Light Exposure Putatively by Targeting Mitochondria, SPIE Proc Vol 2972: Optical Methods for Tumor Treatment and Detection Ed. T. Dougherty, San Jose 1997.
Joane Taylor: Effect of Fluence Rate on Tumor Oxygenation and Vascular Responses to Photodynamic Therapy, INABIS 1998
Bisland S K, Lilge L, Lin A, Rusnov R, Wilson B. C.: Metronomic photodynamic therapy; rationale and preclinical evaluation of technical feasibility for treating malignant brain tumors, Photochem.-Photobiol. (2004) 80 (22-30)
Keith Langmack, Ro Mehta, Paul Twyman, Paul Norris: Topical photodynamic therapy at low fluence rates—theory and practice; Journ. Photochemistry and Photobiology B. Biology (2001) 60 (37-43)
T. M. Busch, E. P. Wileyto and all: Photodynamic Therapy creates Fluence rate—dependent Gradients in the Intratumoral Spatial Distribution of Oxygen, Cancer Research (2002), 62 (7273-7279)
Philip Hahnfeldt, Judah Folkman, Lyn Hlatky: Minimizing long term Tumor Burden; The Logic for Metronomic Chemotherapeutic Dosing and its Antiangiogenic Basis, J. Theor. Biol. (2003), 220 (545-554)
Gianpietro Gasparini: Metronomic scheduling; the future of chemotherapy, The Lancelot Oncology (2001) 2 (733-739)

Detection of Fluorescent Light Using Light Emitting Diodes

A matrix of the red light emitting diodes is used to detect a fluorescent red light generated when the Soret's absorption waveband of ppix has been excited.

A characteristic, that a light emitting diode can be a narrow-band monochromatic photodetector, besides of emitting the monochromatic light, has been illustrated in literature in detail.

Detection by light emitting diodes has been published in the literature:
1. Mims Forrest M III. Sun Photometer with light-emitting diode as spectrally selective detectors, Appl. Opt. (1992), 31, (6965-6967)
2. Y. B. Acharya: Spectral emission characteristic of LED and its applications to LED-based sun-photometry, Optic & Laser Technology, 2005, 37-7-(547-550)
3. Acharya Y. B., Jayaraman A. Ramachadran S. Subbaraya B. H. Compact light emitting diode sun-photometer for atmospheric optical depth measurement (Appl. Opt. (1995), 34-7, (1209-1214)
4. Miyuzuki E., Itami S., Araki T.: Using a light emitting diode as a high speed wavelength selective photodector, Rev. Sci. Instrum. (1998) 69 (II) (3751-3754)
5. Paul Dietz, William Yerazumis, Daren Leigh: Very Low-cost Sensing and Communication Using Bidirectional LEDs: Mitsubishi Electric Research Laboratories Inc.; TR 2003-35, 2003 Broadway, Cambridge, Mass. 02139, July 2003

From the literature and patent review it is evident that:
As a photoreactive agent for photodynamic therapy of skin tumorous diseases protoporphyrin IX has been used
5-aminolevulinic acid and its derivatives have been used to generate protoporphyrin IX
protoporphyrin IX accumulates in tumorous cells
ppix illuminated with the light at the wavelengths of 400 nm to 700 nm generates singlet oxygen and produces a photodynamic effect, that is to say, it selectively destroys tumorous cells.
Illuminated with the light of 400 nm, it fluoresces at the wavelength of 630 nm and this fluorescence gives measure of the concentration of protoporphyrin IX in the tissue, that is, the concentration of the tumorous cells.
A few types of illuminators for photodynamic therapy has been known in literature and patent bases.
Fractional therapy is more efficient than a continuous therapy of the same dose
For illumination, the matrix of light emitting diodes (abbr. LEDs) with the different wavelengths, embedded in transparent plastics or a photodynamic bandage, has been used.

In the literature and patent bases available, it has not been found that:
For photodynamic treatment and diagnostics, a contact sequential illuminator is used, the usage of which is for photodynamic therapy and diagnostics at the same time.
The illuminator is consisted of the two types of light emitting diodes: the red ones with the emission at 640 nm and the violet ones with the emission at the wavelengths of 390 nm to 410 nm

It has not been found that the red light emitting diodes serve for twofold purpose:
1. To emit the red light that serves for administering photodynamic therapy
2. To detect the red fluorescent radiation of ppix that is excited with the violet light emitting diodes.

It has not been found that the red light emitting diodes are driven to illuminate for a certain time and at certain intensity depending on the measured fluorescent radiation of ppix.

It has not been found that so constructed illuminator has a multiple purpose owing to this twofold role of the red light emitting diodes:
1. To monitor the state of the ppix fluorescence, and thereby its concentration during photodynamic therapy
2. That thanks to monitoring the ppix fluorescence, it enables the photodynamic process is performed in an optimal regime in relation to the oxygen passing into the area treated.
3. To illuminate with the red therapeutic light only those areas where the fluorescence does exist, that means it does not illuminate the area of healthy tissue. The illuminator applied in this way is selective spatially, and the photodynamic process does not damage the healthy tissue area.

All these points mentioned that have not been found in literature and patent bases are the subject of this patent.

### DETAILED DESCRIPTION OF THE INVENTION

The main aim of this patent is to establish control and increase the efficiency of the photodynamic therapeutic process. In addition, the aim of this invention is to diminish damage of healthy tissue and reduce pain sensation in the photodynamic procedure.

The essence of the invention is that the level of protoporphyrin IX (ppix) is being monitored during the photodynamic procedure and in relation to that, a dynamics of the process is determined. A concentration level of ppix is determined in relation to its fluorescence intensity. The fluorescence is measured in a matrix that consists of the red light emitting diodes.

The essence of the invention is that the two types of light emitting diodes are used: the violet ones (390 nm-410 nm) which serve for exciting the fluorescence of ppix, and the red ones that have twofold purpose: they serve for therapeutic excitation of ppix and detection of its fluorescent light.

The intelligent sequential illuminator for photodynamic therapy of surface tumors operates by means of the matrix of the red light emitting diodes and the violet light emitting diodes that function sequentially.

A concentration level of ppix decreases during photodynamic therapy, and in relation to it, the intensity of fluorescence also decreases. In the initial period (before illumination), the fluorescence intensity is maximal. This intensity decreases during therapy until is dropped at the minimum value after certain time.

The time, during which the maximum fluorescent intensity (Ifmax) is being decreased at the minimum value (Ifmin), depends on the intensity of the therapeutic light.

After the therapeutic illumination has been stopped, there is recovery of the concentration of ppix so that the fluorescence intensity is increased.

The essence of the invention is to stop with the therapeutic illumination at moment until the fluorescent intensity is dropped at the before determined value. After this, one will wait until the fluorescent intensity (and with that the concentration of ppix, too) reaches a given value. Thereupon, the therapeutic process continues. This process can be repeated until the fluorescent intensity drops at the minimum value

Ifmin after more successive fractional illuminations.

The intelligent sequential illuminator for photodynamic therapy of surface tumors operates in the following manner:
1. The intensity of ppix concentration is being measured in the regime of 5-ALA incubation. When the fluorescent intensity has reached its maximum values, the therapeutic regime starts.
2. The starting fluorescent intensity is being measured on the lesion after incubation with 5-ALA (violet diodes are ON, and the red ones are in the detection mode).
3. The regime of photodynamic therapy starts when maximum fluorescent intensity has been determined. The violet light emitting diodes operate in a given tact: while the violet light emitting diodes are ON, the red ones are in the detection mode. While the red light emitting diodes are ON, the violet ones are OFF.
4. During the violet pulse excitement, the red light emitting diodes measure the intensity of fluorescent radiation.
5. When the intensity of the fluorescent radiation has dropped below the before determined value, it is stopped illuminating with the red light.
6. When the fluorescent intensity has reached the given value, the red light emitting diodes switch ON, and the therapeutic process is repeated.
7. The photodynamic therapeutic process unfolds in a following sequential order:

7.1. The red light emitting diodes switch ON to the emission regime.
7.2. After the time determined, the red light emitting diodes switch OFF the emission regime and turn over into the detection regime.
7.3. Simultaneously with the item 7.2., the violet light emitting diodes switch ON.
7.4. The red light emitting diodes measure the fluorescent intensity caused by the item 7.3.
7.5. A microprocessor decides whether to switch the red light emitting diodes into the emission regime or not, depending on the change of the fluorescent value.
7.6. At the expiration of time, the violet light emitting diodes are ON, the red light emitting diodes get into the detecting mode simultaneously, and the sequence is repeated.
8. The maximum fluorescent signal becomes lower and lower during a repeating period of the sequential order. When the fluorescent signal has reached the before determined value, the matrix with the red light emitting diodes does not switch ON any more. A system operates in a recovering regime. After stopping the illumination with the red light emitting diodes to recover the concentration of ppix, the fluorescent signal starts rising again until its new maximum value is reached. This gives a signal to the processor to switch the red light emitting diodes into the emission regime, and the process is repeated. This recurrence continues until the fluorescent signal drops at the minimal value and after the time determined does not recover any more.

The governing component of the illuminator for the photodynamic therapy of the surface tumors comprises a matrix of the violet light emitting diodes operating at the given tact. The time duration of a pulse of the violet light emitting diodes is short enough so that its illumination dose does not influence the saturation of photo bleaching of ppix.

### DESCRIPTION OF THE DRAWINGS

FIG. 1 represents a block scheme of the device whereby the following designations have the following meaning:
1 Module for illumination and detection
2 Module for signal processing and control
3 Module for power supply
4 Matrix of the red light emitting diodes
5 Matrix of the violet light emitting diodes
6 Amplifier
7 AD converter
8 DA converter
9 Microcontroller, i.e. the module for control
10 User interface

FIG. 2. represents an emission and detection spectral characteristic of the red light emitting diodes and the violet light emitting diodes whereby the designation has the following meaning:
11 Emitting spectral characteristics of the violet light emitting diodes
12 Emitting spectral characteristics of the red light emitting diodes
13 Detection spectral characteristics of the light emitting diodes

FIG. 3. represents the arrangement of the red light emitting diodes and the violet light emitting diodes in the module for illumination and detection whereby the calling sign is:
14 Arrangement of the violet light emitting diodes
15 Arrangement of the red light emitting diodes
16 Arrangement of the resistor of the violet light emitting diodes
17 Printed circuit board

FIG. 4. represents a cross-section of the module for illumination and detection whereby the designation is:
18 Printed circuit board of the violet light emitting diodes
19 Silicone in which the module for illumination and detection is embedded
20 Violet light emitting diodes
21 Resistor of the violet light emitting diodes
22 Printed circuit board of the red light emitting diodes
23 Red light emitting diodes
24 Resistor of the violet light emitting diodes

FIG. 5. represents the sequences of operation of the contact illuminator for photodynamic therapy of surface tumors
I Sequence of the pulses intensities of the violet excited pulses
II Sequence of the measured fluorescent peaks
III Sequence of the peak intensities of the red light emitting diodes

FIG. 6. represents the sequence of the illumination regime from which it is evident that the fluorescence intensity is lower at each further fraction.

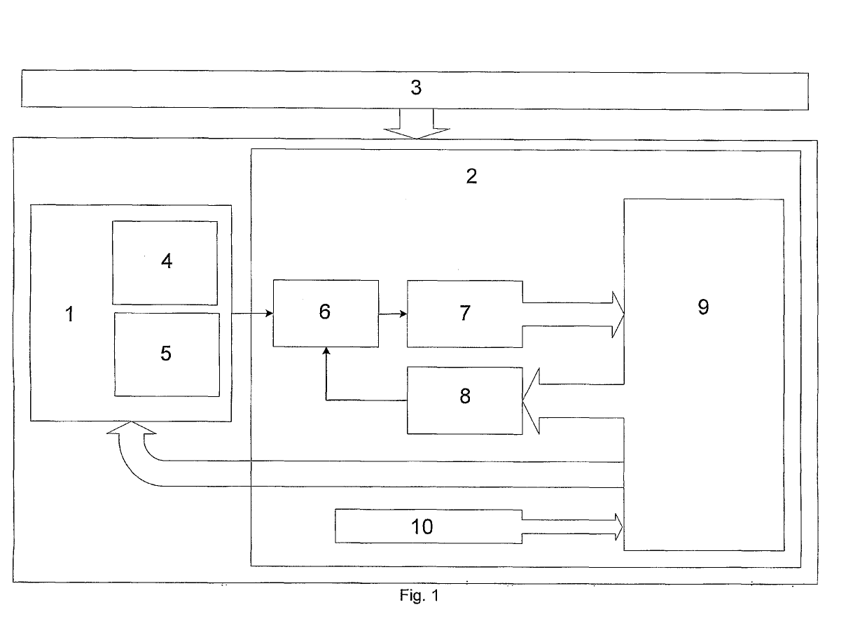

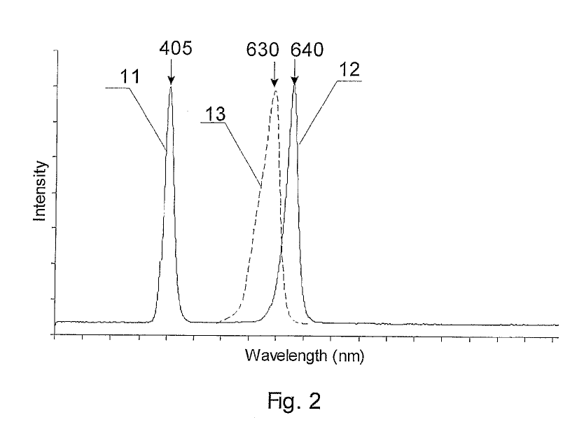

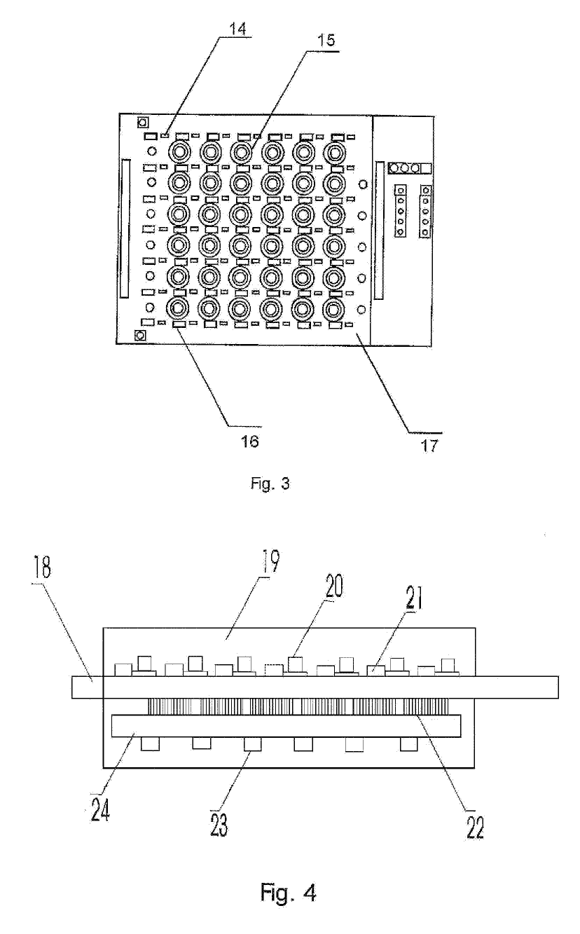

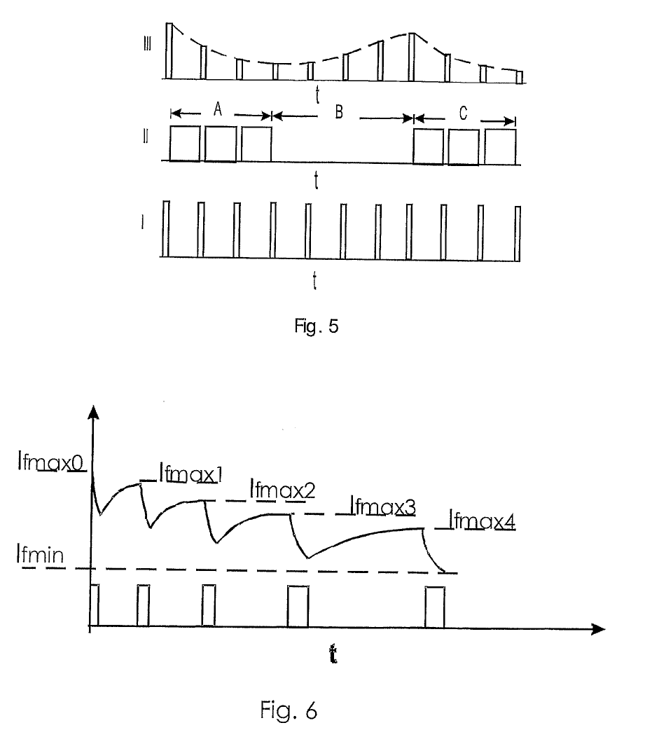

### DESCRIPTION OF THE DEVICE

Intelligent sequential illuminator for photodynamic therapy of surface tumors (FIG. 1) comprises: a module for illumination and detection 1 module for signal processing and control 2 and a module for power supply 3. The module for illumination and detection comprises: a matrix of the red light emitting diodes 4, matrix of the violet light emitting diodes 5 and a housing of the module. The matrix of the red light emitting diodes 4 comprises an array of the red light emitting diodes that emit light at a waveband of about 640 nm. This wavelength is in the range of the red edge of the absorption band of ppix.

In relation to the emission maximum at 640 nm, a detection sensitivity of these diodes is shifted toward shorter wavelengths and is in the area of 630 nm to 635 nm, and this is the area of maximum ppix fluorescence (FIG. 2).

In addition, the selected light emitting diodes emit light at the maximum wavelength that is acceptable for ppix absorption. This maximum wavelength does penetrate the tissue maximally.

By way of selecting the emission wavelength a photodynamic therapy with the maximum penetration is achieved and a detection of maximum fluorescence is obtained. In this way and thanks to this, the same red light emitting diodes are used to emit the therapeutic light and detect the ppix fluorescence. The matrix of the violet light emitting diodes 5 (FIGS. 1 and 4) comprises a network of the light emitting diodes 20 with the pertaining resistors 21. This wavelength is in the range of the maximum band absorption—so called Soret's band. Printed circuit board (PCB) with the red light emitting diodes 23 and the violet light emitting diodes 20 (FIG. 4) is embedded in transparent silicone 19. A silicone thickness 19 from the emitting surfaces of the light emitting diodes is selected so that the uniform distribution intensity of the red light emitting diodes is obtained on the surface. In this case, a homogenous illumination of the region treated is ensured, and the uniform detection of the fluorescent light is achieved.

The module for signal processing and control 2 (FIG. 1) consists of the following modules: a module for analogue signal processing enables to amplify and shape an analogue signal obtained from the red light emitting diodes when these work in a regime of photo-detection. The signal, obtained from the red light emitting diodes when the diodes operate in the detection regime, is very law and therefore is amplified first. A trans-impedance amplifier 6 is used for signal amplifying. A voltage at the amplifier output is converted into the digital signal 7 (FIG. 1). The data obtained are stored in the memory of a microcontroller 9 (in a control module), they are compared with the set parameters, and on the bases of the information so obtained, a decision is made whether to continue with the therapeutic illumination process or not.

The main component of the control module is the microcontroller 9. This module governs the operation of a whole apparatus. A control of the device relates to an activation and deactivation of the light emitting diodes: as those red ones 23 at 640 nm for therapy as well as those violet ones 20 in the range of 395 nm to 410 nm for exciting a fluorescence of ppix.

In addition, the module for control 9 controls the modules for analogue signal processing and serves for communication with a user.

A user interface 10 (FIG. 1) enables to adjust the parameters which determine a course of incubation with 5-ALA and the photodynamic therapy. The module for power supply 3 (FIG. 1) enables the power supply is obtained by means of a battery. Its duty cycle is sufficiently long to perform a fractional therapeutic regime. For a metronomic therapeutic regime several batteries are used which are activated after a definite time. The battery power supply enables the patients are mobile and the device is used ambulatory.

The working method of an electronic system of Intelligent sequential Illuminator for photodynamic therapy of the surface tumors

Intelligent sequential illuminator for photodynamic therapy of the surface tumors operates so that a fluorescence of the exogenous ppix generated by the matrix of the violet light emitting diodes 5 is detected by means of the matrix of the red light emitting diodes 4 (405 nm). A measurement result of the fluorescence intensity so obtained is used to control emission of the red light emitting diodes that emit the red therapeutic light at the wavelength of 640 nm.

A signal of the photocurrents generated through illumination with the violet light emitting diodes 20 that is obtained from the red light emitting diodes 23 when they work in the detection regime consists of 3 components:
1. A photocurrent signal of a parasitic fluorescence that comes from the fluorescence of the material of the light emitting diodes, material with which the matrix of the light emitting diodes is embedded and the fluorescence of other fluorofores, except of ppix in the tissue.
2. A signal of the photocurrents of the endogenous ppix fluorescence that comes from the healthy tissue out of a tumorous lesion. This signal gives information about the condition of the tissue and accumulation of ppix in the healthy tissue. It is a referent signal that determines a lower limit of the maximum fluorescence signal in the diseased tissue. It is measured on the healthy tissue and is stored in a memory.
3. A photocurrent fluorescence signal of the endogenous ppix in a tumorous lesion. This signal depends on an accumulation rate of the exogenous ppix in the tumorous lesion. It is changed during photodynamic therapy process and is essential for the dynamics of illumination.

It is supposed that the signals of the parasitic fluorescence and those ones of the endogenous ppix are constant during photodynamic process. These two signals are treated as one parasitic signal and they are stored together in the memory. The signal 13 of the fluorescence of the exogenous ppix is essential for regulation of the photodynamic therapeutic procedure. A signal of the joint parasitic signal is subtracted at the input of the amplifier in order to increase the amplifier dynamics. In the photodynamic therapy process, the data of the exogenous fluorescence ppix and the data of the parasitic fluorescence are converted into the analogous signal by means of the digital-analogous converter. This analogous signal is brought into a second input of the amplifier and is subtracted from the signal of the exogenous fluorescence ppix of the tissue diseased. Herewith, only a component coming from the fluorescence of the exogenous ppix of the tissue diseased is obtained at the output of the amplifier. The so amplified signal converts into a digital form, and is stored in the memory. On the bases of the measured signal, the microcontroller controls an activation-deactivation process of the red light therapeutic diodes 23.

The therapeutic process stops working when the maximum intensity of the exogenous fluorescence of PpIX drops below a determined value. Herewith, the intelligent sequential illuminator for photodynamic therapy of surface tumors is disconnected.

7. A Way in which the Invention is Applied

The invention “Intelligent sequential illuminator for photodynamic therapy of skin surface tumors” enables an efficient and reliable photodynamic therapy of skin benign and malignant tumorous diseases. This invention enables essential improvements in relation to the previous photodynamic illuminators. Photodynamic therapy with this invention is very simple. After 5-ALA cream has been put onto the tumorous lesion, a transparent bandage is placed, and then the contact illuminator for surface tumors is placed onto the bandage that is covered with a non-transparent bandage. After the illuminator has been switched on, it works in the regime of incubation, an increase in ppix accumulation in the tissue is being measured by means of fluorescence. When the fluorescence reaches its maximum, a process of incubation is finished. The time, needed to accomplish that, can last from 2 to 6 hours. Upon the time expiration, a therapeutic process begins. The therapeutic process activates a sequential working of the violet light emitting diodes and the red light emitting diodes. After certain numbers of sequences, the therapeutic illumination—recurrence of the ppix concentration, the device disconnects by itself, signalizing that the photodynamic process is completed. The Intelligent sequential illuminator for photodynamic therapy of surface tumors can be used in an ambulance. After installing the device, a patient is sent home. When the illuminator for photodynamic therapy of surface tumors signalizes that the therapeutic procedure is finished, the patient himself can remove the device and store it.

With regard that the intensity of the therapeutic illumination with the intelligent illuminator for photodynamic therapy of surface tumors is considerably lower then the former ones, the level of pain or discomfort, which occurs at the photodynamic therapy, is also lower. If a patient feels pain, the patient himself can switch off the device and turn it on again when the pain sensation is gone.

1. Intelligent sequential illuminating device comprising a module for illumination and detection, module for signal processing and control and module for power supply, characterized in that the said module for illumination and detection consists of two kinds of matrices of light emitting diodes: the matrix with the light emitting diodes of short wavelength serving to excite the fluorescence of photoreactive agent and the matrix with the led emitting diodes of longer wavelength where the said matrix with the light emitting diodes of longer wavelength has twofold purpose: the first purpose is to provide the detection of fluorescent light of the photoreactive agent excited by the said matrix with the light emitting diodes of shorter wavelength and the second purpose is to provide the optimal excitation of the photoreactive agent in the sense that the said module with the matrix of the shorter wavelength and the said module with the matrix of the longer wavelength are sequentially activated with the different activation times of the said matrix with the longer wavelength according to the measured fluorescence intensity levels of the photoreactive agent measured by the same matrix with the light emitting diodes of the longer wavelength whereby the said intensity level depends on the photoreactive agent concentration.

2. The illuminator according to claim 1, characterized in that, the matrix of the red light emitting diodes comprises an array of the red light emitting diodes which detects at the wavelength of the maximum fluorescence of the photo reactive agent.

3. The illuminator according to claim 1, characterized in that, the red light emitting diode emit at the maximum wavelength that is acceptable for absorption of the photo reactive agent, wherewith it is enabled the light emission and detection of the fluorescence of the photo reactive agent.

4. The illuminator according to claim 1, characterized in that, the matrix of the light emitting diodes comprises an array of the light emitting diodes with the resistors emitting in the wave length needed to excite the fluorescence of the photo reactive agent.

5. The illuminator according to claim 1, characterized in that, the matrix of the violet light emitting diodes and the red light emitting diodes together with the module for analog signal processing and the module for control estimate a concentration level of the photoreactive agent in the period of the photoreactive agent accumulation and in relation to the fluorescence intensity an optimum time of incubation.

6. The illuminator according to claim 1, characterized in that, the red light emitting diodes stop illuminating by means of the matrix of the violet light emitting diodes, and the matrix of the red light emitting diodes, and the system of the microprocessor logic module, when the fluorescent signal caused by means of the violet light emitting diodes drops below the determined level, i.e. switches on the matrix array of the red light emitting diodes when the concentration of the photo reactive agent has recurred, i.e. until the concentration of the photo reactive agent drops at the minimum before determined value.
